(19) United States
(12) Patent Application Publication
(10) Pub. No.: US 2019/0375724 A1
(43) Pub. Date: Dec. 12, 2019

(54) PROCESS FOR PREPARING A CYCLIC DIESTER OR A CYCLIC DIAMIDE BY REACTING A HYDROXYCARBOXYLIC ACID OR AMIDE WITH AN ACIDIC BEA-TYPE (H-BETA POLYMORPH A) ZEOLITE

(71) Applicant: BASF SE, Ludwigshafen am Rhein (DE)

(72) Inventors: Alvaro GORDILLO, Heidelberg (DE); Andrei-Nicolae PARVULESCU, Ludwigshafen (DE); Henelyta SANTOS RIBEIRO, Ludwigshafen (DE); Joerg ROTHER, Heidelberg (DE); Ivana JEVTOVIKJ, Heidelberg (DE); Ulrich MUELLER, Ludwigshafen (DE); Stefan MAURER, Shanghai (CN)

(73) Assignee: BASF SE, Ludwigshafen am Rhein (DE)

(21) Appl. No.: 16/485,291

(22) PCT Filed: Mar. 14, 2018
(86) PCT No.: PCT/EP2018/056382

§ 371 Date: Aug. 12, 2019

(30) Foreign Application Priority Data
Mar. 15, 2017 (EP) 17161096.7

(51) Int. Cl. C07D 319/12

(52) U.S. Cl. CPC C07D 319/12; B01J 29/7007

(57) ABSTRACT

A process for preparing a cyclic diester or a cyclic diamide by reacting a hydroxycarboxylic acid or amide with an acidic BEA (H-beta polymorph A) type zeolite. The process is characterised in that the total amount of acid sites is in the range of from 0.25 to 1.0 mmol/g and the amount of medium acid sites is at least 40% of the total amount of acid sites. The total amount of acid sites and the amount of medium acid sites are determined by NH3-TPD (temperature-programmed desorption of ammonia). Preferably, the process refers to the preparation of lactide from lactic acid. The framework structure of the zeolitic material comprises Si, Al, O, and H.

The present invention relates to a process for preparing cyclic esters and cyclic amides using a catalyst comprising a zeolitic material having framework type BEA.

Cyclic esters are compounds that can be polymerized into polymeric materials that are useful in the preparation plastic materials such as plastic materials. Cyclic esters can also be used as plasticizers and as intermediates for production of surface-active agents and plasticizers.

WO 2014/122294 A discloses the preparation of cyclic ester by contacting hydroxycarboxylic acid with acidic zeolite.

There is the need for processes for preparing cyclic esters and amides which are economically advantageous and can be carried out with high selectivity, high conversion and high yield.

The present invention therefore relates to a process for preparing a cyclic esters and cyclic amides of formula (II)

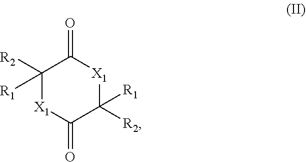

comprising

(i) providing a mixture comprising a compound of formula (I)

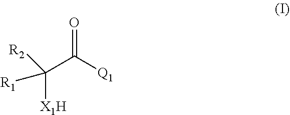

or a salt thereof;

(ii) contacting the mixture provided in (i) with a catalyst comprising a zeolitic material, obtaining a mixture (ii) comprising the compound of formula (II); wherein

X₁ is O or NH;

R₁ and R₂ are, independently of each other, H, C₁-C₁₀ alkyl, C₂-C₁₀ alkenyl, C₂-C₁₀ alkynyl, or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;

Q₁ is OH, OR₃, NH₂, Cl, Br, or I;

R₃ is C₁-C₁₀ alkyl or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;

wherein the zeolitic material in (ii) has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.; and wherein the amount of medium acid sites is at least 40% of the total amount of acid sites.

The term “C₁-C₁₀ alkyl” as used in the context of the present invention refers to an alkyl residue having from 1 to 10 carbon atoms in the chain. The alkyl residue may have, for example, 1, 2, 3, 4, 5,or 6 carbon atoms in the chain (C₁-C₆ alkyl) or 1, 2, 3, or 4 carbon atoms in the chain (C₁-C₄ alkyl). The alkyl residue may be a linear or a branched alkyl residue. The alkyl residue includes, but not is limited to, methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, and n-octyl. The alkyl residue can be optionally substituted.

The term “C₂-C₁₀ alkenyl” as used in the context of the present invention refers to an alkenyl residue having from 2 to 10 carbon atoms in the chain. The alkenyl residue may have, for example, 2, 3, 4, 5,or 6 carbon atoms in the chain (C₂-C₆ alkenyl) or 2, 3 or 4 carbon atoms in the chain (C₂-C₄ alkenyl). The alkenyl residue may be a linear or a branched alkenyl residue. The alkenyl residue includes, but not is limited to, ethenyl, 2-propenyl, 2-butenyl, 3-butenyl, 2-pentenyl and its chain isomers, 2-hexenyl and 2,4-pentadienyl. The alkenyl residue can be optionally substituted.

The term “C₂-C₁₀ alkynyl” as used in the context of the present invention refers to an alkynyl residue having from 2 to 10 carbon atoms in the chain. The alkynyl residue may have, for example, 2, 3, 4 or 6 carbon atoms in the chain (C₂-C₆ alkynyl) or 2, 3 or 4 carbon atoms in the chain (C₂-C₄ alkynyl). The alkynyl residue may be a linear or a branched alkynyl residue. The alkynyl residue can be optionally substituted.

The term “C₁-C₆ alkyloxy” as used in the context of the present invention refers to an alkyloxy residue having from 1 to 6 carbon atoms in the chain. The alkyloxy may have, for example, 2, 3, 4, 5 or 6 carbon atoms in the chain (C₂-C₆ alkyloxy) or 2, 3 or 4 carbon atoms in the chain (C₂-C₄ alkyloxy). The alkyloxy residue may be a linear or a branched alkyloxy residue. The alkyloxy residue includes, but not is limited to, methoxy, ethoxy, propoxy, isopropoxy, butoxy, isobutoxy, sec-butoxy, ter-butoxy, pentyloxy, and hexyloxy. The alkyloxy residue can be optionally substituted.

The term “C₆-C₁₂ aryl” as used in the context of the present invention refers to an aromatic residue having from 6 to 12 carbon atoms. The aryl residue includes, but not to be limited to, phenyl, naphtyl, indanyl, and 1,2,3,4-tetrahydronaphthyl.

The term “optionally substituted” as used in the context of the present invention is to be understood to include any suitable substituent conceivable for the skilled person to be comprised in the compound of formula (I) which does not prevent the formation of the compound of formula (II) according to the present process.

According to the invention, X₁ is preferably O.

Preferably, R₁ is H, methyl, ethyl, propyl, isopropyl, n-butyl, or ethenyl, and R₂ is H, methyl, ethyl, propyl, isopropyl, n-butyl, or ethenyl. More preferably, R₁ is H, and R₂ is H, methyl, ethyl, propyl, isopropyl, or ethenyl. More preferably, R₁ is H, R₂ is H or CH₃, X₁ is O, and Q₁ is H. More preferably, R₁ is H, R₂ is CH₃, X, is O, and Q₁ is H.

In the compound of formula (I) the carbon bearing R₁ and R₂ is a stereogenic center provided that R₁ is different from R₂. The stereogenic center can have configuration S or R according to the Cahn Ingold and Prelog (CIP) nomenclature.

In the process of the invention it is further preferred that compound of formula (I) is one or more of

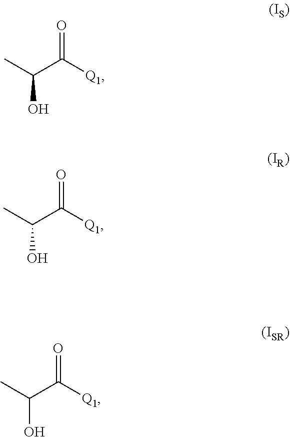

wherein (I_{S}) is the S enantiomer, (I_{R}) is the R enantiomer and (I_{SR}) is the racemate. The compound, without specific reference to the stereochemistry, is known as lactic acid. The compound of formula (I_{S}) is the S enantiomer of the lactic acid and is also known as L-lactic acid. Preferably, the compound of formula (I) is the compound of formula (I_{S}). The compound of formula (II) is preferably one or more of

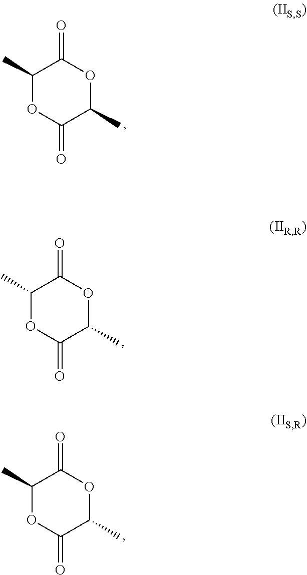

and the racemate of (II_{S,S}) and (II_{R,R}). Without specific reference to the stereochemistry this compound is known as 3,6-dimethyl-1,4-dioxan-2,5 dione.

In the process of the invention it is more preferred the compound of formula (II) is the compound of formula (II_{S,S})

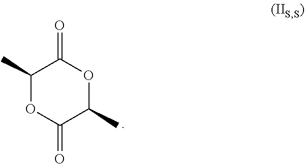

Hence, the present invention is preferably directed to a process for preparing 3,6-dimethyl-1,4-dioxan-2,5 dione, the process comprises
(i) providing a mixture comprising 2-hydroxypropanoic acid (lactic acid);
(ii) contacting the mixture provided in (i) with a catalyst comprising a zeolitic material, obtaining a mixture (ii) comprising 3,6-dimethyl-1,4-dioxan-2,5-dione,

wherein the zeolitic material in (ii) has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.; and

wherein the amount of medium acid sites is at least 40% of the total amount of acid sites.

In the process of the invention, when X₁ is NH, the compounds of formula (I) is preferably one aminoacid such as alanine, glycine, leucine, valine. The aminoacid can be in the pure enantiomeric form S or R, preferably in the S form or in the racemic form.

It has been found that when the catalyst of the invention is used in a condensation reaction, advantageously no racemization at the stereogenic center occurs. The present reaction hence occurs with high enantioselectivity or in other word with an enantiomeric excess of at least 90%. The enantiomeric excess %ee is herein defined as % ee=([E1)−(E2)/(E1)+(E2)]×100 wherein E1 and E2 refer to the molar amount of the two enantiomers.

Step (i)

In step (i) of the process according to the invention, a mixture comprising the compound of formula (I) is provided.

Preferably, the mixture comprising the compound of formula (I) comprises water. No particular limitation exists as to the amount of the compound of formula (I) in the mixture relative to the water amount. Preferably, in the mixture provided in (i), the weight ratio of the compound of formula (I) relative to the water is in the range of from 95:5 to 45:55, more preferably in the range of from 95:5 to 85:15 or in the range of from 45:55 to 55:45.

It is further preferred that at least 80 weight-%, more preferably at least 85 weight-%, more preferably at least 90 weight-%, more preferably at least 95 weight-% of the mixture of (i) consists of the compound of formula (I) and of water wherein the weight-% is based on the total weight of the mixture.

It is generally conceivable that reaction according to the present invention is carried out in batch mode or in semi-continuous mode or in continuous mode. It is preferred that the reaction is carried out in continuous mode. The reaction can be carried out in a liquid phase or in a gaseous phase. It is preferred that the reaction is carried out in a liquid phase. It is further more preferred that the reaction is carried out in continuous mode and in liquid phase.

It is conceivable that when the reaction is carried out in a liquid phase, the reaction is carried out in a solvent. It is further conceivable that the catalyst of (ii) is comprised in a solvent and that the mixture provided in (i) and the catalyst of (ii) is comprised in a solvent are brought together. For example, in a batch mode the mixture of (i) and the catalyst comprised in a solvent are brought together in a reactor. According to the continuous mode, it is possible that the mixture of (i) in liquid phase, and preferably the solvent, in liquid form, are passed into a suitable reaction zone. Prior to passing the mixture of (i) and the solvent into the reaction zone, i.e. prior to (ii), the mixture of (i) and the solvent can be admixed with each other.

No particular restriction exists with respect to the solvent provided that the compound of formula (II) is formed. The solvent is chosen also in dependence of the temperature of the reaction. The solvent further preferably forms an azeotropic mixture with water or is immiscible with water. Water may come from the mixture of (i) and water is formed during the reaction. Water needs to be removed from the reaction in (ii). It is hence preferred that the solvent is an organic solvent suitable for an easy removal of water from the reaction. It is further preferred that the organic solvent is one or more of an aromatic solvent, aliphatic (open chain) solvent, cyclic hydrocarbon solvent, ethers. More preferably, the solvent comprises, more preferably consists of, one or more of pentane, hexane, heptane, petroleum ether, cyclohexane, dichloromethane, trichloromethane, tetrachloromethane, benzene, toluene, xylene, chlorobenzene, dichlorobenzene, diethylether, methyl-tert-butylether, dibutylether, tetrahydrofuran, dioxane, acetonitrile, propionitrile. More preferably, the solvent is an aromatic solvent, more preferably the aromatic solvent comprises, more preferably consists of, one or more of benzene, toluene and xylene.

As to the amount of the compound of formula (I) relative to the organic solvent no particular limitation exist. Preferably in the the mixture provided in (i), the molar ratio of the compound of formula (I) relative to the organic solvent is in the range of from 0.01:1 to 3:1, more preferably in the range of from 0.05:1 to 2:1, more preferably in the range of from 0.1:1 to 1:1.

Hence according to the present invention the mixture of (i) comprises the compound of formula (I), the organic solvent and water. Preferably at least 95 weight-%, more preferably at least 98 weight-%, more preferably at least 99 weight-% of the mixture provided in (i) and subjected to (ii) consist of the compound of formula (I), the organic solvent, and water.

Water is preferably removed under the reaction conditions of step (ii). Preferably, the water content of the mixture provided in (i) and subjected to (ii) is at most 5 weight-%, more preferably at most 1 weight-%, more preferably at most 0.1 weight-%.

Hence according to the present invention a process is preferably provided for preparing the compound of formula (II), preferably for preparing 3,6-dimethyl-1,4-dioxan-2,5-dione, the process comprising
(i) providing a mixture comprising the compound of formula (I) as defined above, preferably providing a mixture comprising 2-hydroxypropanoic acid (lactic acid);
(ii) contacting the mixture provided in (i) with a catalyst comprising a zeolitic material, obtaining a mixture (ii) comprising the compound of formula (II) preferably 3,6-dimethyl-1,4-dioxan-2,5-dione,

wherein the zeolitic material in (ii) has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.;

wherein the amount of medium acid sites is at least 40% of the total amount of acid sites; and

wherein the contacting of (H) is carried out in the presence of an organic solvent, wherein the organic solvent is preferably one or more of an aromatic solvent, and wherein preferably the aromatic solvent is one or more of benzene, toluene or xylene. The organic solvent can be added to the mixture of (i) before the contacting of (ii) or the zeolitic material can be comprised in the organic solvent and the mixture of (i) is added to the organic solvent which comprised the zeolitic material.

The reaction can also be carried out in gaseous phase, wherein the compound of formula (I), in gaseous form, and a diluent, in gaseous form, and optionally a carrier gas in a gaseous form are brought into contact with the catalyst according to the invention.

No specific restrictions exist with regard to the chemical nature of the diluent. It is preferred that the diluent is an organic solvent. Preferably the organic solvent is one or more of an aromatic solvent, aliphatic (open chain) solvent, cyclic hydrocarbon solvent, ethers. More preferably, the solvent comprises, more preferably consists of, one or more of pentane, hexane, heptane, petroleum ether, cyclohexane, dichloromethane, trichloromethane, tetrachloromethane, benzene, toluene, xylene, chlorobenzene, dichlorobenzene, diethylether, methyl-tert-butylether, dibutylether, tetrahydrofuran, dioxane, acetonitrile, propionitrile. More preferably, the solvent is an aromatic solvent, more preferably the aromatic solvent comprises, more preferably consists of, one or more of benzene, toluene and xylene. More preferably, the solvent does not comprise water.

No specific restrictions exist with regard to the chemical nature of the carrier gas. Preferably, the carrier gas is a gas or a mixture of two or more gases which is inert with respect to the reaction. The term “inert” as used in this context of the present invention relates to a gas or a mixture of two or more gases which does not have a negative influence on the reaction. Preferably, the carrier gas comprises one or more of helium, argon, nitrogen, more preferably nitrogen. More preferably, the carrier gas is nitrogen, more preferably technical nitrogen having a nitrogen content of at least 99.5 volume-% and an oxygen content of at most 0.5 volume-%.

With regard to the amount of carrier gas used, no specific restrictions exist, and the volume ratio of the carrier gas relative to the compound of formula (I) can be varied in wide ranges. Preferably, prior to contacting the compound of formula (I) with the catalyst the volume ratio of the carrier gas relative to the compound of formula (I) in its gaseous form is in the range of from 1:1 to 20:1, more preferably in the range of from 2:1 to 15:1, more preferably in the range of from 5:1 to 10:1.

Step (ii)

The Zeolitic Material

The zeolitic material used in the process of the invention is a zeolitic material having framework structure of type BEA. Preferably, the zeolitic material according to the invention is an organotemplate-free zeolitic material having framework structure of type BEA. The expression “organotemplate-free zeolitic material having framework structure of type BEA” according to the invention means that in the process for the preparation of said zeolite no more than an impurity of an organic structure directing agent specifically used in the synthesis of zeolitic materials having a BEA-type framework structure, in particular specific tetraalkylammonium salts and/or related organotemplates such as tetraethylammonium and/or dibenzylmethylammonium salts, and dibenzyl-1,4-diazabicyclo[2,2,2]octane is present. Such an impurity can, for example, be caused by organic structure directing agents still present in seed crystals used in the inventive process. Organotemplates contained in seed crystal material may not, however, participate in the crystallization process since they are trapped within the seed crystal framework and therefore may not act structure directing agents.

Typically, zeolitic materials have acid sites that are Broensted acid sites. In particular the zeolitic material of the invention has Broensted acid sites. The acid sites present in the zeolite material can have different acidic strength. Accordingly the acid sites with reference to the acidic strength are named as medium acid sites or strong acid sites. The total amount of acid sites as herein defined is the total molar amount of desorbed ammonia per mass of the calcinated zeolitic material as measured according to the temperature programmed desorption of ammonia (NH3-TPD) method as disclosed in Reference Example 1.1. The zeolitic material of the invention further comprises a certain amount of medium acid sites, or a certain amount of medium acid sites and a certain amount of strong acid sites. The amount of medium acid sites as herein defined is the amount of desorbed ammonia per mass of the zeolitic material as measured according to the temperature programmed desorption of ammonia method in the temperature range of from 250 to 500° C. The amount of strong acid sites as herein defined is the amount of desorbed ammonia per mass of the zeolitic material as measured according to the temperature programmed desorption of ammonia method at a temperature above 500° C.

As to the amount of medium acid sites and strong acid sites there is no particular limitation provided that the compound of formula (II) is formed. According to the present invention the amount of medium acid sites is preferably in the range of from 40 to 70%, more preferably in the range of from 50 to 70%, more preferably in the range of from 60 to 70% of the total amount of acid sites. In terms of the molar amount of medium acid sites, no particular limitation exists provided that the compound of formula (II) according to the invention is formed. It is preferred that the molar amount of medium acid sites of the zeolitic material of the invention is in the range of from 0.10 to 0.60 mmol/g. More preferably the molar amount of medium acid sites of the zeolitic material of the invention is in the range of from 0.20 to 0.50 mmol/g. The amount of acid sites in the zeolitic material of the invention is determined according to the temperature programmed desorption of ammonia (NH3-TPD) method as disclosed in Reference Example 1.1.

In terms of the amount of strong acid sites no particular limitation exists provided the above mentioned amount of medium acid is fulfilled and the compound of formula (II) according to the invention is formed. The amount of strong acid sites of the zeolitic material is defined as the amount of desorbed ammonia per mass of the zeolitic material determined according to the temperature programmed desorption of ammonia (NH₃-TPD) as described in Reference Example 1.1 herein in the temperature range above 500° C. Preferably, the amount of strong acid sites is in the range of from 0 to 0.10 mmol/g, more preferably is in the range of from 0 to 0.07 mmol/g, more preferably in the range of from 0 to 0.04 mmol/g.

In terms of the amount of total acid sites, no particular limitation exists provided that compound of formula (II) according to the invention is formed. As to the total amount of acid sites in the zeolitic material according to the present invention it is preferred that it is in the range of from 0.25 to 1.0 mmol/g, preferably the total amount of acid sites is in the range of from 0.40 to 0.60 mmol/g wherein the amount is determined according to the temperature programmed desorption of ammonia (NH3-TPD) method as disclosed in Reference Example 1.1.

According to the invention further the ratio of the amount of medium acid sites relative to amount of strong acid sites is defined. Preferably, the ratio of the amount of medium acid sites relative to amount of strong acid sites is greater than 0, more preferably said ratio is at least 10:1, more preferably said ratio is at least 20:1.

Hence the present invention is preferably direct to process for preparing 3,6-dimethyl-1,4-dioxan-2,5-dione, preferably the process of any one of embodiments 1 to 46, comprising
(i) providing a mixture comprising 2-hydroxypropanoic acid;
(ii) contacting the mixture provided in (i) with a catalyst comprising a zeolitic material, obtaining a mixture (ii) comprising 3,6-dimethyl-1,4-dioxan-2,5-dione,

wherein the zeolitic material in (ii) has framework type BEA and wherein the framework structure of the zeolitic material comprises Si, Al, O and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.;

wherein the amount of medium acid sites is at least 40% of the total amount of acid sites and

wherein the amount of medium acid sites in the range of from 0.10 to 0.60 mmol/g, preferably in the range of from 0.20 to 0.50 mmol/g. Preferably, according to the invention, compound 3,6-dimethyl-1,4-dioxan-2,5-dione is (35,65)3,6-dimethyl-1,4-dioxan-2,5-dione.

As to the framework structure of the zeolitic material, it comprises Si, Al, O, and H. According to the present invention there is no limitation with regard to the molar ratio Si:Al, provided that the compound of formula (II) is formed. Preferably according to the invention the framework structure of the zeolitic material has molar ratio Si:Al in the range of from 15:1 to 30:1, more preferably in the range of from 20:1 to 25:1.

The zeolitic material of the invention can be used in the form of a powder i.e. as such or it can be formulated with binders. Hence, the catalyst of (ii) may further comprise, in addition to the zeolitic material, one or more binders. It is preferred that the zeolitic material of the invention is used in the form of a powder without the addition of a binder. When a binder is used, it is preferred that the binder is one or more one or more of graphite, silica, titania, zirconia, a mixture of oxides of two or more of Si, Ti, and Zr, and a mixed oxide of two or more of Si, Ti, and Zr.

It is further preferred that the weight ratio of the zeolitic material relative to the binder material is in the range of from 10:1 to 3:1. More preferably, the weight ratio of the zeolitic material relative to the binder material is in the range of from 9:1 to 4:1.

According to the invention, there is no limitation as to the form of the catalyst of the invention.

Preferably the catalyst in (ii) is in the form of a powder or in the form of a shaped body, wherein the shaped body preferably has a rectangular, a triangular, a hexagonal, a square, an oval or a circular cross section, and/or is in the form of a star, a tablet, a sphere, or a hollow cylinder. More preferably, the catalyst in (ii) is in the form of a powder.

As mentioned above in step (i) it is generally conceivable that contacting the compound of formula (I) with the catalyst is carried out in batch mode or in semi-continuous mode or in continuous mode. It is preferred that the contacting is carried out in continuous mode. The reaction can be carried out in a liquid phase or in a gaseous phase. It is preferred that the reaction is carried out in a liquid phase. It is more preferred that the reaction is carried out in continuous mode and in liquid phase.

Hence the contacting of (ii) is preferably carried out in the present of a solvent. As mentioned above, the solvent can be provided in the mixture of (i) or the solvent can comprise the zeolitic material of the invention and being brought into contact with the mixture of (i). The solvent is as defined above in “Step (i)”.

As mentioned above, water may be comprised in the mixture of (i). Further, water is formed during the reaction. The water needs to be removed from the reaction solvent in order the reaction to be optimally carried out. It has been seen that the presence of water reduces the reaction conversion, because the reaction is an equilibrium reaction which depends on the amount of water. Hence, the process as disclosed above preferably is carried out in conditions of water removal. Water can be removed by one or more of azeotropic distillation, evaporation, molecular sieve, water absorbing material such as silica or polysaccharides or anhydrous material. Preferably, water is removed via azeotropic distillation. According to the invention it is preferred that the water content of the mixture provided in (i) and subjected to (ii) is at most 5 weight-%, more preferably at most 1 weight-%, more preferably at most 0.1 weight-%.

Hence, the contacting of the compound of formula (I) with the catalyst is preferably carried out at a temperature of the liquid phase which is preferably at least 100° C., more preferably in the range of from 100 to 250° C., more preferably in the range of from 120 to 200° C., more preferably in the range of from 130 to 170° C. The absolute pressure of the liquid phase at which said contacting is carried out is preferably in the range of from 2 to 10 bar_{(abs)}, more preferably in the range of from 0.5 to 5 bar_{(abs)}, more preferably in the range of from 0.75 to 2 bar_{(abs)}. Therefore, preferably, the contacting of the compound of formula (II) with the catalyst is carried out at a temperature of the liquid phase in the range of at least 100° C., preferably in the range of from 100 to 250° C., more preferably in the range of from 120 to 200° C., more preferably in the range of from 130 to 170° C. and an absolute pressure of the gas phase in the range of from 2 to 10 bar_{(abs)}, preferably in the range of from 0.5 to 5 bar_{(abs)}, more preferably in the range of from 0.75 to 2 bar_{(abs)}. Therefore, it is more preferred that the contacting of the compound of formula (II) with the catalyst is carried out at a temperature of the liquid phase in the range of from 130 to 170° C. and an absolute pressure of the gas phase in the range of from 0.75 to 2 bar_{(abs)}.

As to the space velocity (weight hourly space velocity, WHSV) with respect to the contacting in (ii) of the process according to the invention, it is preferably chosen such that an advantageous balance of conversion, selectivity, yield, reactor geometry, reactor dimensions and process regime is obtained. In the context of the present invention, the weight hourly space velocity is defined as the mass flow of the compound of formula (I) comprised in the mixture provided in (i) an subjected to (ii) in kg/h divided by the mass of the zeolitic material comprised in the catalyst in kg with which the mixture provided in (i) is contacted in (ii). The space velocity therefore has the unit (1/time). Preferably, the WHSV in the present process is in the range of from 0.5 to 10 h ⁻¹, more preferably in the range of from 1.5 to 5 h⁻¹.

Further Steps

The valuable products of formula (II) obtained in (ii) can be separated from the mixture of (ii) according to generally known methods, including extraction, distillation, crystallization or chromatographic isolation. Therefore, the present invention also relates to the process as described above, wherein said process further comprising separating the compound of formula (II) from the mixture of (ii).

Further, the process according to the invention may additionally comprise the regenerating of the catalyst used in (ii). In this context, it may be conceivable to regenerate the catalyst at a temperature elevated relative to room temperature in a suitable gas atmosphere for a suitable period of time. Further, the process according to the invention may additionally comprise the recycling of the compound of formula (I) which may be present in non-converted form in the mixture obtained from (ii). Preferably the recycling the compound of formula (I) is in to the process according to the present invention.

Further, the process according to the invention, wherein the mixture obtained in (ii) further comprises the organic solvent, may additionally comprise recycling the organic solvent, preferably recycling the organic solvent to the process of the invention.

Process for Preparing the Zeolitic Material of the Invention

As mentioned above, the preferred zeolitic materials of the invention are organotemplate-free zeolitic materials having framework structure of type BEA. Methods for preparing organotemplate-free zeolitic material having framework structure of type BEA are known in the art.

Hence, the present invention is directed to a process for preparing a compound of formula (II) wherein the zeolitic material comprised in the catalyst according to (ii) is obtainable or obtained by an organotemplate-free synthesis method.

Hence, the present invention is directed to a process for preparing a compound of formula (II), the process further comprising preparing the zeolitic material comprised in the catalyst according to (ii) by an organotemplate-free synthesis method.

A method for preparing organotemplate-free zeolitic material having framework structure of type BEA is for example disclosed in patent application WO 2010/146156 A. This process comprises
(1) providing a mixture comprising one or more sources for SiO₂, one or more sources for Al₂O₃, and seed crystals, wherein the seed crystals comprise a zeolitic material having framework type BEA;
(2) crystallizing the mixture obtained in step (1), obtaining a mixture comprising the zeolitic material having a framework type BEA; and
(3) isolating the zeolitic material having framework type BEA from the mixture obtained from (2); and
(4) preferably drying and calcining the zeolitic material having framework type BEA.

According to this process for preparing the organotemplate-free zeolitic material, SiO₂ can be provided in step (1) in any conceivable form, provided that a zeolitic material having a BEA framework structure comprising SiO₂ can be crystallized in step (2). Preferably, SiO₂ is provided as such and/or as a compound which comprises SiO₂ as a chemical moiety and/or as a compound which (partly or entirely) is chemically transformed to SiO₂ during the process. The source for SiO₂ provided in step (1) can be any conceivable source. There can therefore be used, for example, all types of silica and silicates, preferably fumed silica, silica hydrosols, reactive amorphous solid silicas, silica gel, silicic acid, water glass, sodium metasilicate hydrate, sesquisilicate or disilicate, colloidal silica, pyrogenic silica, silicic acid esters, or tetraalkoxysilanes, or mixtures of at least two of these compounds. The source of SiO₂ preferably comprises at least one compound selected from the group consisting of silica and silicates, preferably silicates, more preferably alkali metal silicates. Among the preferred alkali metal silicates, the at least one source preferably comprises water glass, more preferably sodium and/or potassium silicate, and more preferably sodium silicate. In particular preferably the source for SiO₂ is sodium silicate. In particular, when the at least one source for SiO₂ comprises water glass, crystallization is accelerated. This especially applies when water glass is the only source for SiO₂ used in the process for preparing the zeolitic material of the invention.

The source for Al₂O₃ provided in step (1) can be any conceivable source. There can be used for example any type of alumina and aluminates, aluminum salts such as, for example, alkali metal aluminates, aluminum alcoholates, such as, for example, aluminum triisopropylate, or hydrated alumina such as, for example, alumina trihydrate, or mixtures thereof. Preferably, the source for Al₂O₃ comprises at least one compound selected from the group consisting of alumina and aluminates, preferably aluminates, more preferably alkali metal aluminates. Among the preferred alkali metal aluminates, the at least one source preferably comprises sodium and/or potassium aluminate, more preferably sodium aluminate.

The preferred zeolitic material according to the invention is prepared according to the above process comprising steps (1) to (3), preferably according to the above process comprising steps (1) to (4). Hence, the present invention is preferably directed to a process for preparing a compound of formula (II) as disclosed above, wherein the zeolitic material is obtained or is obtainable according to the process as disclosed above comprising steps (1) to (3), preferably according to the process as disclosed above comprising steps (1) to (4).

Hence, according to the present invention, a process is preferably provided for preparing the compound of formula (II), preferably for preparing 3,6-dimethyl-1,4-dioxan-2,5-dione, wherein the process comprises
(i) providing a mixture comprising the compound of formula (I) as defined above, preferably comprising 2-hydroxypropanoic acid (lactic acid);
(ii) contacting the mixture provided in (i) with a catalyst comprising a zeolitic material, obtaining a mixture (ii) comprising the compound of formula (II) preferably 3,6-dimethyl-1,4-dioxan-2,5-dione,

wherein the zeolitic material in (ii) is an organotemplate-free zeolitic and has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.; and

wherein the amount of medium acid sites is at least 40% of the total amount of acid sites.

Hence according to the present invention, a process is further preferably provided for preparing the compound of formula (II), preferably for preparing 3,6-dimethyl-1,4-dioxan-2,5-dione, wherein the process comprises
(i) providing a mixture comprising the compound of formula (I) as defined above, preferably comprising 2-hydroxypropanoic acid (lactic acid);
(ii) contacting the mixture provided in (i) with a catalyst comprising a zeolitic material, obtaining a mixture (ii) comprising the compound of formula (II), preferably 3,6-dimethyl-1,4-dioxan-2,5-dione,

wherein the zeolitic material in (ii) is an organotemplate-free zeolitic and has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.;

wherein the amount of medium acid sites is at least 40% of the total amount of acid sites and

wherein the process comprises preparing the organotemplate-free zeolitic material of (ii) according to a process comprising
(1) providing a mixture comprising one or more sources for SiO₂, one or more sources for Al₂O₃, and seed crystals, wherein the seed crystals comprise a zeolitic material having framework type BEA;
(2) crystallizing the mixture obtained in step (1), obtaining a mixture comprising the zeolitic material having a framework type BEA; and
(3) isolating the zeolitic material having framework type BEA from the mixture obtained from (2); and
(4) preferably drying and calcining the zeolitic material having framework type BEA.

Steps (1) to (4) are carried out preferably according to the conditions disclosed in patent applications WO2010146156.

Therefore, a preferred zeolitic material according to the invention is a zeolitic material that has been prepared according to the process comprising steps (1) to (3), preferably steps (1) to (4). Hence, the present invention is preferably directed to a process for preparing a compound of formula (II) as disclosed above, wherein the zeolitic material is obtained or is obtainable according to the process as disclosed above comprising steps (1) to (3), preferably steps (1) to (4). Hence, the present invention is preferably directed to a process for preparing a compound of formula (II) as disclosed above, further comprising the process as disclosed above comprising steps (1) to (3), preferably steps (1) to (4).

The zeolitic material of the invention, preferably the organotemplate-free zeolite obtained according to the above process, can further be subjected to a post-treatment such as acid treatment and stream treatment or combination thereof. A preferred zeolitic material according to the invention has been subjected to a post-treatment, preferably has been subjected to the post treatment disclosed in patent application WO 2014/060260 A. The process involves subjecting a zeolitic material to at least one treatment with an aqueous solution having a pH of at most 5 and at least one treatment with a liquid aqueous system having a pH in the range of 5.5 to 8 at elevated temperatures of at least 75° C. The treatment removes or partially removes the Al element. However, although the Al element is partially removed from the zeolitic material during said post-treatment process, the resulting zeolitic material exhibits high crystallinity and even a reduced concentration of internal defects. Therefore, the process for preparing the zeolitic material as disclosed above further comprises a post-treatment of the zeolitic material according to the steps:
(5) subjecting the zeolitic material obtained from (3) or (4), preferably from (4), to a method comprising
   (5.1) treating the zeolitic material with an aqueous solution having a pH of at most 5;
   (5.2) treating the zeolitic material obtained from (5.1) with a liquid aqueous system having a pH in the range of 5.5 to 8 and a temperature of at least 75° C.;
   wherein after (5.2), the zeolitic material is optionally subjected to at least one further treatment according to (5.1) and/or at least one further treatment according to (5.2); and
   wherein the pH of the aqueous solution according to (5.1) and the pH of the liquid aqueous system according to (5.2) is determined using a pH sensitive glass electrode.

Therefore, a preferred zeolitic material according to the invention is a zeolitic material that has been subjected to a post treatment as disclosed above, preferably a post treatment according to the process comprising steps (5.1) and (5.2). Hence, the present invention is preferably directed to a process for preparing a compound of formula (II) as disclosed above, wherein the zeolitic material is obtained or is obtainable according to the process as disclosed above comprising steps (1) to (5). Hence, the present invention is preferably directed to a process for preparing a compound of formula (II) as disclosed above, further comprising the process as disclosed above comprising steps (1) to (5).

Hence, according to the present invention a process is preferably provided for preparing the compound of formula (II), preferably for preparing 3,6-dimethyl-1,4-dioxan-2,5-dione, wherein the process comprises
(i) providing a mixture comprising the compound of formula (I) as defined above, preferably comprising 2-hydroxypropanoic acid (lactic acid);
(ii) contacting the mixture provided in (i) with a catalyst comprising a zeolitic material, obtaining a mixture (ii) comprising the compound of formula (II) preferably 3,6-dimethyl-1,4-dioxan-2,5-dione,

wherein the zeolitic material in (ii) is an organotemplate-free zeolitic and has framework type BEA and wherein the framework structure of the zeolitic material comprises Si, Al, O, and H;

wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;

wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.;

wherein the amount of medium acid sites is at least 40% of the total amount of acid sites;

and wherein the process comprises preparing the zeolitic material of (ii) according to a process comprising
(1) providing a mixture comprising one or more sources for SiO₂, one or more sources for Al₂O₃, and seed crystals, wherein the seed crystals comprise a zeolitic material having framework type BEA;
(2) crystallizing the mixture obtained in step (1), obtaining a mixture comprising the zeolitic material having a framework type BEA; and
(3) isolating the zeolitic material having framework type BEA from the mixture obtained from (2);
(4) preferably drying and calcining the zeolitic material having framework type BEA;
(5) subjecting the zeolitic material obtained from (3) or (4), preferably from (4), to a method comprising
   (5.1) treating the zeolitic material with an aqueous solution having a pH of at most 5;
   (5.2) treating the zeolitic material obtained from (5.1) with a liquid aqueous system having a pH in the range of 5.5 to 8 and a temperature of at least 75° C.;
   wherein after (5.2), the zeolitic material is optionally subjected to at least one further treatment according to (5.1) and/or at least one further treatment according to (5.2);
   and wherein the pH of the aqueous solution according to (a) and the pH of the liquid aqueous system according to (b) is determined using a pH sensitive glass electrode.

The present invention is further directed to a mixture comprising a compound of formula (II)

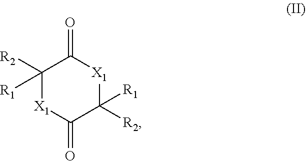

preferably a compound of formula (II_{S,S})

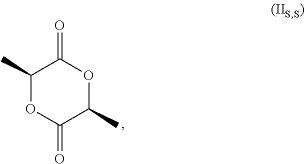

wherein the mixture is obtainable or obtained by any one of the processes as disclosed herein above.

The present invention is further directed to the use of a zeolitic material as defined above, as a catalytically active material in an esterification and/or in an amidation reaction. The starting material of the esterification and/or amidation reaction is preferably the compound of formula (I) as disclosed above. The product of the esterification and/or amidation reaction is preferably the compound of formula (II) as disclosed above.

The present invention is further directed to a method for preparing an ester and/or an amide, wherein the product of said method is preferably a compound of formula (II) as disclosed above. The method according to the invention uses the organotemplate-free zeolitic material having a BEA-type framework structure as disclosed herein.

The present invention is further directed to the use of the compound of formula (II) as defined above, optionally comprised in the mixture obtained in (ii), as a cyclic dimer starting material for preparing an oligomer or a polymer.

The present invention is further illustrated by the following embodiments and combinations of embodiments as indicated by the respective dependencies and back-references. In particular, it is noted that if a range of embodiments is mentioned, for example in the context of a term such as “The process of any one of embodiments 1 to 4”, every embodiment in this range is meant to be disclosed for the skilled person, i.e. the wording of this term is to be understood by the skilled person as being synonymous to “The process of any one of embodiments 1, 2, 3, and 4”.
1. A process for preparing a compound of formula (II)

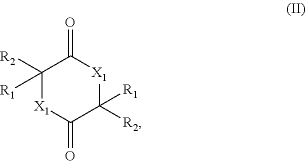

comprising
(i) providing a mixture comprising a compound of formula (I)

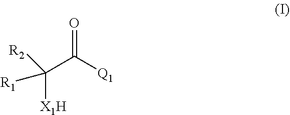

or a salt thereof;
(ii) contacting the mixture provided in (i) with a catalyst comprising a zeolitic material, obtaining a mixture (ii) comprising the compound of formula (II);
wherein
X₁ is O or NH;
R₁ and R₂ are, independently of each other, H, C₁-C₁₀ alkyl, C₂-C₁₀ alkenyl, C₂-C₁₀ alkynyl, or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;
Q₁ is OH, OR₃, NH₂, Cl, Br, or I;
R₃ is C₁-C₁₀ alkyl or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;
wherein the zeolitic material in (ii) has framework type BEA and wherein the framework structure of the zeolitic material comprises Si, Al, O, and H;
wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;
wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C.;
wherein the amount of medium acid sites is at least 40% of the total amount of acid sites.
2. The process of embodiment 1, wherein the amount of medium acid sites is in the range of from 40 to 70%, preferably in the range of from 50 to 70%, more preferably in the range of from 60 to 70% of the total amount of acid sites.
3. The process of embodiment 1 or 2, wherein the amount of medium acid sites is in the range of from 0.10 to 0.60 mmol/g.
4. The process of any one of embodiments 1 to 3, wherein the amount of medium acid sites is in the range of from 0.20 to 0.50 mmol/g.
5. The process of any one of embodiments 1 to 4, wherein the amount of strong acid sites of the zeolitic material defined as the amount of desorbed ammonia per mass of the zeolitic material determined according to the temperature programmed desorption of ammonia (NH₃-TPD) as described in Reference Example 1.1 herein in the temperature range above 500° C. is in the range of from 0 to 0.10 mmol/g.
6. The process of embodiment 5, wherein the amount of strong acid sites is in the range of from 0 to 0.07 mmol/g, preferably in the range of from 0 to 0.04 mmol/g.
7. The process of any one of embodiments 1 to 6, wherein the ratio of the amount of medium acid sites relative to amount of strong acid sites is greater than 0.
8. The process of any one of embodiments 1 to 7, wherein the ratio of the amount of medium acid sites relative to amount of strong acid sites is at least 10:1, preferably at least 20:1.
9. The process of any one of embodiments 1 to 8, wherein the framework structure of the zeolitic material has a molar ratio Si:Al in the range of from 15:1 to 30:1, preferably in the range of from 20:1 to 25:1.
10. The process of any one of embodiments 1 to 9, wherein the zeolitic material comprised in the catalyst according to (ii) is obtainable or obtained by an organotemplate-free synthesis method.
11. The process of any one of embodiments 1 to 9, further comprising preparing the zeolitic material comprised in the catalyst according to (ii) by an organotemplate-free synthesis method.
12. The process of embodiment 10 or 11, wherein the organotemplate-free synthesis method comprises
   (1) providing a mixture comprising one or more sources for SiO₂, one or more sources for Al₂O₃, and seed crystals, wherein the seed crystals comprise a zeolitic material having framework type BEA;
   (2) crystallizing the mixture obtained in step (1), obtaining a mixture comprising the zeolitic material having a framework type BEA; and
   (3) isolating the zeolitic material having framework type BEA from the mixture obtained from (2).
   (4) preferably drying and calcining the zeolitic material having framework type BEA.
13. The process of embodiment 12, wherein the method comprises
   (5) subjecting the zeolitic material obtained from (3) or (4), preferably from (4), to a method comprising
      (5.1) treating the zeolitic material with an aqueous solution having a pH of at most 5;
      (5.2) treating the zeolitic material obtained from (5.1) with a liquid aqueous system having a pH in the range of 5.5 to 8 and a temperature of at least 75° C.;
   wherein after (5.2), the zeolitic material is optionally subjected to at least one further treatment according to (5.1) and/or at least one further treatment according to (5.2).
14. The process of any one of embodiments 1 to 13, wherein the catalyst in (ii) is in the form of a powder or in the form of a shaped body, wherein the shaped body preferably has a rectangular, a triangular, a hexagonal, a square, an oval or a circular cross section, and/or is in the form of a star, a tablet, a sphere, or a hollow cylinder.
15. The process of embodiment 14, wherein the catalyst in (ii) is in the form of a powder.
16. The process of any one of embodiments 1 to 14, wherein the catalyst in (ii) is in the form of a shaped body and comprises a binder material in addition to the zeolitic material, wherein the binder material is preferably one or more of graphite, silica, titania, zirconia, a mixture of oxides of two or more of Si, Ti, and Zr, and a mixed oxide of two or more of Si, Ti, and Zr.
17. The process of embodiment 16, wherein the weight ratio of the zeolitic material relative to the binder material is in the range of from 10:1 to 3:1, preferably in the range of from 9:1 to 4:1.
18. The process of any of embodiments 1 to 17, wherein the mixture provided in (i) further comprises water.
19. The process of embodiment 18, wherein in the mixture provided in (i), the weight ratio of the compound of formula (I) relative to the water is in the range of from 95:5 to 45:55, preferably in the range of from 95:5 to 85:15 or in the range of from 45:55 to 55:45.
20. The process of any one of embodiments 1 to 19, preferably of embodiment 19, wherein the mixture provided in (i) further comprises an organic solvent.
21. The process of embodiment 20, wherein the organic solvent forms an azeotropic mixture with water.
22. The process of embodiment 21, wherein the organic solvent is immiscible with water.
23. The process of any one of embodiments 20 to 22, wherein the organic solvent is one or more of an aromatic solvent, an aliphatic (open chain) solvent, a cyclic hydrocarbon solvent, an ether, preferably one or more of pentane, hexane, heptane, petroleum ether, cyclohexane, dichloromethane, trichloromethane, tetrachloromethane, benzene, toluene, xylene, chlorobenzene, dichlorobenzene, diethylether, methyl-tert-butylether, dibutylether, tetrahydrofuran, dioxane, acetonitrile, and propionitrile, wherein more preferably, the organic solvent is one or more of benzene, toluene and xylene.
24. The process of any one of embodiments 20 to 23 wherein in the mixture provided in (i), the molar ratio of the compound of formula (I) relative to the organic solvent is in the range of from 0.01:1 to 3:1, preferably in the range of from 0.05:1 to 2:1, more preferably in the range of from 0.1:1 to 1:1.
25. The process of any one of embodiments 20 to 24, wherein at least 95 weight-%, preferably at least 98 weight-%, more preferably at least 99 weight-% of the mixture provided in (i) and subjected to (ii) consist of the compound of formula (I), the organic solvent, and water.
26. The process of any one of embodiments 25, wherein the water content of the mixture provided in (i) and subjected to (ii) is at most 5 weight-%, preferably at most 1 weight-%, more preferably at most 0.1 weight-%.
27. The process of any one of embodiments 1 to 26, wherein the mixture provided in (i) is provided in liquid phase.
28. The process of any one of embodiments 1 to 27, wherein according to (ii), the mixture provided in (i) is contacted with the catalyst in liquid phase or in gas phase, preferably in liquid phase.
29. The process of any one of embodiments 1 to 28, insofar as being dependent on embodiment 19 or 20, wherein the contacting of (ii) is carried out under water removal conditions, wherein the water removal conditions preferably comprise an azeotropic distillation.
30. The process of any one of embodiments 1 to 29, wherein the contacting in (ii) is carried out at a temperature of the mixture brought in contact with the catalyst of at least 100° C., preferably in the range of from 100 to 250° C., more preferably in the range of from 120 to 200° C., more preferably in the range of from 130 to 170° C.
31. The process of any one of embodiments 1 to 30, wherein the contacting in (ii) is carried out at a pressure of the mixture brought in contact with the catalyst in the range of from 2 to 10 bar_{(abs)}, preferably in the range of from 0.5 to 5 bar_{(abs)}, more preferably in the range of from 0.75 to 2 bar_{(abs)}.
32. The process of any one of embodiments 1 to 31, wherein the contacting in (ii) is carried out at a weight hourly space velocity in the range of from 0.5 to 10 h⁻¹, preferably in the range of from 1.5 to 5 h⁻¹, wherein the weight hourly space velocity is defined as the mass flow rate of the compound of formula (I) comprised in the mixture provided in (i) and subjected to (ii) in kg/h divided by the mass of the zeolitic material comprised in the catalyst in kg with which the mixture provided in (i) is contacted in (ii).
33. The process of any one of embodiments 1 to 32, wherein the contacting in (ii) is carried out in batch mode or in semi-continuous mode or in continuous mode, preferably in continuous mode.
34. The process of any one of embodiments 1 to 33, further comprising separating the compound of formula (II) from the mixture obtained from (ii).
35. The process of any one of embodiments 1 to 34, wherein the mixture obtained in (ii) further comprises the compound of formula (I), the process further comprising recycling the compound of formula (I), preferably recycling the compound of formula (I) to the process according to any one of embodiments 1 to 34.
36. The process of any one of embodiments 1 to 35, insofar being dependent on embodiment 20, wherein the mixture obtained in (ii) further comprises the organic solvent, the process further comprising recycling the organic solvent, preferably recycling the organic solvent to the process according to any one of embodiments 1 to 35.
37. The process of any one of embodiments 1 to 36, further comprising regenerating the catalyst.
38. The process of any one of embodiments 1 to 37, wherein C₁-C₁₀-alkyl is methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methyl pentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, or n-octyl.
39. The process of any one of embodiments 1 to 38, wherein C₂-C₆ alkenyl is ethenyl, 2-propenyl, 2-butenyl, 3-butenyl, 2-pentenyl and its chain isomers, 2-hexenyl or 2,4-pentadienyl.
40. The process of any one of embodiments 1 to 39, wherein C₂-C₆ alkynyl is ethynyl, 2-propynyl, 2-butynyl, 3-butynyl, 2-pentynyl, or 2-hexynyl.
41. The process of any one of embodiments 1 to 40, wherein C₆-C₁₂ aryl is phenyl, naphthyl, indanyl, or 1,2,3,4-tetrahydro-naphthyl.
42. The process of any one of embodiments 1 to 41, wherein C₁-C₆ alkyloxy is methoxy, ethoxy, propoxy, isopropoxy, butoxy, isobutoxy, sec-butoxy, tert-butoxy, pentyloxy, or hexyloxy.
43. The process of any one of embodiments 1 to 42, wherein R₁ is H, R₂ is H, X1 is O, and Q₁ is OH.
44. The process of any one of embodiments 1 to 42, wherein R₁ is H, R₂ is CH₃, X1 is O, and Q₁ is H.
45. The process of any one of embodiments 1 to 44, wherein the compound of formula (I) is one or more of

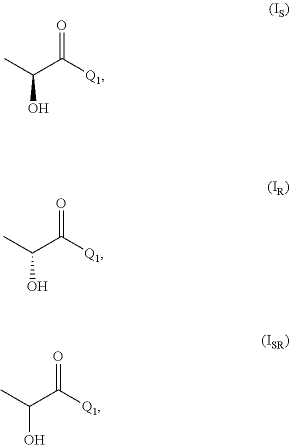

preferably the compound of formula (I_{S}), and wherein the compound of formula (II) is preferably one or more of

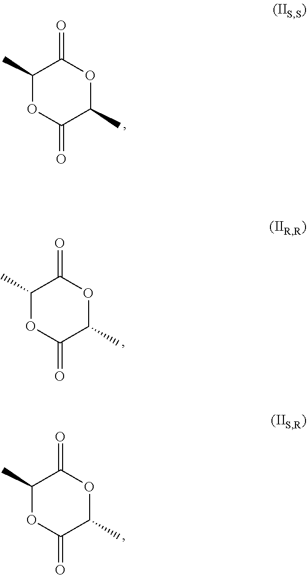

and the racemate of (II_{S,S}) and (II_{R,R}).
46. The process of embodiment 44 or 45, wherein the compound of formula (II) is the compound of formula (II_{S,S})

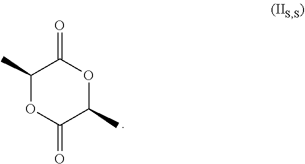

47. A process for preparing 3,6-dimethyl-1,4-dioxan-2,5-dione, preferably the process of any one of embodiments 1 to 46, comprising
   (i) providing a mixture comprising 2-hydroxypropanoic acid;
   (ii) contacting the mixture provided in (i) with a catalyst comprising a zeolitic material, obtaining a mixture (ii) comprising 3,6-dimethyl-1,4-dioxan-2,5-dione,
   wherein the zeolitic material in (ii) has framework type BEA and wherein the framework structure of the zeolitic material comprises Si, Al, O and H;
   wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia (NH3-TPD) as described in Reference Example 1.1 herein;
   wherein the zeolitic material has an amount of medium acid sites wherein the amount of medium acid sites is defined as the amount of desorbed ammonia per mass of the zeolitic material determined according to the temperature programmed desorption of ammonia (NH3-TPD) as described in Reference Example 1.1 herein in the temperature range of from 250 to 500° C., wherein the amount of medium acid sites in the range of from 0.10 to 0.60 mmol/g, preferably in the range of from 0.20 to 0.50 mmol/g.
48. The process of embodiment 47, wherein the compound 3,6-dimethyl-1,4-dioxan-2,5-dione is (3S,6S)3,6-dimethyl-1,4-dioxan-2,5-dione.
49. A mixture comprising a compound of formula (II)

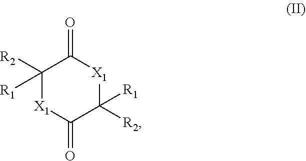

preferably a compound of formula (II_{S,S})

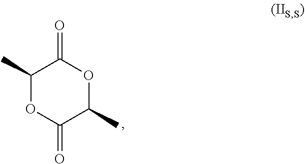

said mixture being obtainable or obtained by a process according to any one of embodiments 1 to 48.
50. Use of a zeolitic material as defined in any one of embodiments 1 to 13 as a catalytically active material in an esterification and/or in an amidation reaction, wherein the product of said reaction is a compound of formula (II) as defined in any one of embodiments 1 to 48.
51. A method for preparing an ester and/or an amide, wherein the ester is preferably a compound of formula (II) as defined in any one of embodiments 1 to 48, said method comprising employing a zeolitic material as defined in any one of embodiments 1 to 13 as a catalytically active material.

The present invention is further illustrated by the following reference examples, examples, and comparative examples.

### EXAMPLES

### Reference Example 1

### Analytical Methods

### Reference Example 1.1

### Determination of the Acid Sites: Temperature Programmed Desorption of Ammonia (NH3-TPD)

The temperature-programmed desorption of ammonia (NH3-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).
2. Saturation with NH₃: Commencement of recording; one measurement per second. Change the gas flow to a mixture of 10% NH₃ in He (75 cm³/min; 100° C. and 1 atm) at 100° C.; hold for 30 min.
3. Removal of the excess: Commencement of recording; one measurement per second. Change the gas flow to a He flow of 75 cm³/min (100° C. and 1 atm) at 100° C.; hold for 60 min.
4. NH₃-TPD: Commencement of recording; one measurement per second. Heat up under a He flow (flow rate: 30 cm³/min) to 600° C. at a heating rate of 10 K/min; hold for 30 min.
5. End of measurement.

Desorbed ammonia was measured by means of the online mass spectrometer, which demonstrated that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Reference Example 1.2

### Analysis of the Mixture Obtained in (ii)

Lactic acid conversion and lactide yield were calculated by ¹H-NMR analysis in DMSO-d6 as described in Dusselier et al., Supplementary Materials for Shape-selective zeolite catalysis for bioplastics production, specifically in section “Reaction analysis”, pages 3-6.

### Reference Example 2

### Preparing the Zeolitic Materials

### Reference Example 2.1

### Preparing a Zeolitic Material having Framework Type BEA, Molar Si:Al Ratio 24:1 and a Total Amount of Acid Sites of 0.41 mmol/g

a) Preparation of a Zeolite having BEA Framework Structure (H-Beta Zeolite)
a1) 335.1 g of NaAlO₂ were dissolved in 7314 g of H₂O while stirring, followed by addition of 74.5 g of zeolite Beta seeds (commercially available from Zeolyst International, Valley Forge, Pa. 19482, USA, under the tradename CP814C, CAS Registry Number 1318-02-1). The mixture was placed in a 20 L autoclave and 7340 g sodium waterglass and 1436 g Ludox® AS40 were added. Crystallization of the obtained aluminosilicate gel took place at 120° C. for 117 h. After having let the reaction mixture cool to room temperature, the solid was separated by filtration, repeatedly washed with distilled water and then dried at 120° C. for 16 h.
a2) 1000 g zeolitic material prepared according to al) were added to 10 g of a 10 weight-% solution of ammonium nitrate. The suspension was heated to 80° C. and kept at this temperature under continuous stirring for 2 h. The solid was filtered hot (without additional cooling) over a filter press. The filter cake was then washed with distilled water (room temperature wash water) until the conductivity of the wash water was below 200 microSiemens/cm. The filter cake was dried for 16 h at 120° C. This procedure was repeated once, affording ion exchanged crystalline product BEA in its ammonium form. A following calcination step at 500° C. for 5 h (heat ramp 1 K/min) afforded ion exchanged crystalline product BEA in its H-form.

b) First Acidic Dealumination

Materials Used:

| | |
|---|---|
| 17.15 kg | H-beta-Zeolite according to a): Si = 34%; Al = 6.3%; Na 0.07%. |
| 51.45 kg | HNO₃ (aq.; 4 weight-%) |

A stirred vessel was charged with 51.45 kg of a solution of HNO₃ (4 weight-%). 17.15 kg of H-beta zeolite were added. The obtained suspension was stirred at 60° C. for 2 h. After cooling to 50° C., the suspension was transferred to a filter press, filtered with a pressure of 3.2 bar and washed for 9 h with 5,484 L of distilled water. The zeolite was dried for 20 h at 120° C. 15.912 kg of zeolite were obtained. This zeolite was calcined in a recirculated muffle furnace by raising the temperature at a rate of 1 K/min to 600° C. for 5 h. 15.889 kg of a white solid were obtained. Elementary analysis: Si=35.5%; Al=4.9%; Na=0.05%.

c) Water Treatment

Materials Used:

| | |
|---|---|
| 15.889 kg | acid treated H-beta-zeolite |
| 127 kg | distilled H₂O |

The zeolite was suspended in a vessel in distilled water. The suspension was heated to 90° C. and stirred for 9 h. The suspension was filtered off on a filter press and dried. The drying was carried out at 120° C. for 68 h. 15.547 kg of H-beta-zeolite were obtained.

d) Second Acidic Dealumination

Materials Used:

| | |
|---|---|
| 15.538 kg | H-beta zeolite of c) |
| 46.614 kg | HNO₃ (aq.; 4 weight-%) |

A vessel was charged with 46.614 kg of a solution of HNO₃ (4 weight-%). 1 5.538 kg of the H-beta zeolite were added. The obtained suspension was stirred at 60° C. for 2 h. After cooling to 50° C., the suspension was transferred to a filter press, filtered with a pressure of 3.2 bar and washed for 3.5 h with 2,114 L of distilled water. The zeolite was dried for 48 h at 120° C. 14.14 kg of zeolite were obtained. This zeolite was calcined in a recirculated muffle furnace with the raising the temperature at a rate of 1 K/min to 600° C. for 5 h. 14.479 kg of a white solid (H-beta zeolite were obtained. Elementary analysis: Si=37.5%; Al=3.8%; Na=0.03%.

e) Water Treatment

Materials Used:

| | |
|---|---|
| 14.479 kg | H-beta zeolite of d) |
| 116 kg | distilled H₂O-dest. |

14.479 kg of H-beta zeolite of were suspended in a vessel in distilled water. The solution was heated to 90° C. and stirred for 9 h. The suspension was filtered off on a filter press and dried. The drying was carried out at 120° C. for 22 h. 14.65 kg of H-beta zeolite were obtained.

f) Third Acidic Dealumination

Materials Used:

| | |
|---|---|
| 13.65 kg | H-beta zeolite of e) |
| 40.95 kg | HNO₃ (aq., 4 weight-%) |

A vessel was charged with 40.95 kg of a solution of HNO₃ (4 weight-%). 13.65 kg of H-beta zeolite of e) were added. The obtained suspension was stirred at 60° C. for 2 h. After cooling to 50° C., the suspension was transferred to a filter press, filtered with a pressure of 3.2 bar and washed for 16.5 h with 1,442 L of distilled water. The zeolite was dried for 68 h at 120° C. 12.658 kg of zeolite were obtained. This zeolite was calcined in a recirculated muffle furnace by raising the temperature at a rate of 1 K/min to 600° C. for 5 h. 12.83 kg of a white solid were obtained. Elementary analysis: Si=37.5%; Al=3.8%; Na=0.03%.

g) Water Treatment

Materials Used:

| | |
|---|---|
| 12.82 kg | H-beta zeolite of f) |
| 103 kg | distilled H₂O |

12.82 kg of H-beta zeolite of f) were suspended in a vessel in distilled water. The suspension was heated to 90° C. and stirred for 9 h. The suspension was filtered off on a filter press and dried. The drying was carried out at 120° C. for 22 h. 12.73 kg of H-beta zeolite were obtained.

h) Fourth Acidic Dealumination

Materials Used:

| | |
|---|---|
| 12.72 kg | H-beta zeolite of g) |
| 38.16 kg | HNO₃ (aq., 8 weight-%) |

A vessel was charged with 38.16 kg of an aqueous solution of HNO₃ (8%). 12.72 kg of H-beta zeolite of g) were added. The obtained suspension was stirred at 60° C. for 2 h. After cooling to 50° C., the suspension was transferred to a filter press, filtered with a pressure of 3.2 bar and washed for 5 h with 1,055 I of distilled water. The zeolite was dried for 25 h at 120° C. 11.802 kg of zeolite were obtained. This zeolite was calcined in a recirculated muffle furnace with raising the temperature at a rate of 1 K/min to 600° C. for 5 h. 11.852 kg of a white solid were obtained. Elementary analysis: Si=42.0%; Al=1.8%; Na<0.01%.

i) Water Treatment

Materials Used:

| | |
|---|---|
| 11.842 kg | H-beta zeolite of h) |
| 95 kg | distilled H₂O |

11.842 kg of H-beta zeolite of h) were suspended in a vessel in distilled water. The suspension was heated to 90° C. and stirred for 9 h. The suspension was filtered off on a filter press and dried. The drying was carried out at 120° C. for 22 h. 11.512 kg of H-beta zeolite were obtained.

j) Fifth Acidic Dealumination

Materials Used:

| | |
|---|---|
| 11.502 kg | H-beta zeolite of i) |
| 34.506 kg | HNO₃ (aq., 15 weight-%) |

A vessel was charged with 34.506 kg of an aqueous solution of HNO₃ (15 weight-%). 11.502 kg of H-beta zeolite of i) were added. The obtained suspension was stirred at 60° C. for 2 h. After cooling to 50° C., the suspension was transferred to a filter press, filtered with a pressure of 3.2 bar and washed for 2.25 h with 1,114 L of distilled water. The zeolite was dried for 24 h at 120° C. This zeolite was calcined in a recirculated muffle furnace with raising the temperature at a rate of 1 K/min to 600° C. for 5 h. 11.097 kg of a white solid were obtained. The obtained zeolitic material has a crystallinity of 73% Elementary analysis: Si=43.5%; Al=1.7%; Na<0.01%.

An NH₃-TPD analysis as disclosed in Reference Example 1.1 was performed on the zeolite of j). The respective NH₃-TPD plot is shown in FIG. 1: no peak is observed at a temperature above 500° C. which indicates that no strong acid site is present in the zeolite. A peak was observed at 328.9° C. which indicates the presence of medium acid sites in the zeolite. The peak observed at a temperature of 177.8° C. relates to weak acid sites. The integration of the curve between the temperatures of 100° C. and 550° C. gives the amount of the total acid sites.

Total amount of acid sites: 0.41 mmol/g, as determined according to the NH₃-TPD method described in Reference Example 1.1.

Total amount of medium acid sites: 0.23 mmol/g, as determined according to the NH₃-TPD method described in Reference Example 1.1.

Total amount of strong acid sites: none detected according to the NH3-TPD method described in Reference Example 1.1.

### Reference Example 2.2

### Preparing a Zeolitic Material having Framework Type BEA, a Molar Si:Al Ratio of 22:1 and a Total Amount of Acid Sites of 0.994 mmol/g

a) Preparation of an Al-Beta Zeolite

Materials Used:

| | |
|---|---|
| tetraethylammnium hydroxide (TEAOH) (aq, 35 weight-%) | 905.70 g |
| NaOH (platelets) | 19.80 g |
| aluminum sulfate (Al₂(SO₄)₃*18 H₂O) | 78.60 g |
| Colloidal silica, Ludox ® AS 40 (40 weight-% silica) | 760.80 g |

Under stirring at 300 r.p.m., a solution of 905.7 g TEAOH and 19.8 g NaOH was prepared in a vessel. After 20 min, 78.6 g aluminum sulfate were suspended therein, followed by stirring for 14 h. Under stirring, 760.8 g Ludox® were added to the obtained solution, followed by stirring for 30 min at 300 r.p.m. The liquid gel, having a pH >14, was then transferred to an autoclave and heated under autogenous pressure to a temperature of 170° C. and kept at that temperature for 24 h under stirring at 150 r.p.m. The obtained material was then separate from its mother liquor by filtration and then subjected to washing. The thus obtained zeolitic material was dried under air for 30 h at 95° C. in a vacuum oven (yield: 536 g) and then calcined at 500° C. for 5 h under air (heating ramp: 2 K/min). Analytics of the obtained material: Al 1.9 weight-%; TOC <0.1 weight-%; Na 3.3 weight-%; BET specific surface area (according to DIN 66131) 562 m²/g.

b) Treatment with Ammonium Nitrate

Materials Used:

| | | |
|---|---|---|
| Distilled water | (3x) | 1350.0 g |
| Ammonium nitrate (NH₄NO₃) 99% | (3x) | 150.0 g |
| Al-beta zeolite (powder) from a) | | 150.0 g |

The zeolite was prepared via ion exchange from the Na-form to the H-Form. 1,500 g of an aqueous ammonium nitrate (10 weight-%) solution was prepared from distilled water and ammonium nitrate. While stirring, 150 g of Al-Beta-Zeolite were added at a pH of 2.5 and heated to 80° C. for 2 h. After cooling, the solution was filtered off and washed to neutral pH with 4 L of distilled water. The entire ion exchange process was then repeated twice. The finally obtained zeolite was dried for 5 h at 120° C. and calcined at 500° C. for 5 h. 123 g of a calcined beta zeolite in its H-form were obtained. Elementary analysis: Al=1.9%; Si=43%; Na=0.01%.

An NH₃-TPD analysis as disclosed in Reference Example 1.1 was performed. The NH₃-TPD plot is shown in FIG. 2: a weak peak was observed at a temperature of 594.3° C. which indicates that a small amount of strong acid sites is present in the zeolite. A peak was observed at 374.1° C. which indicates the presence of medium acid sites in the zeolite. The peak observed at a temperature of 223.8° C. relates to weak acid sites. The integration of the curve between the temperatures of 100° C. and 600° C. gave the amount of the total acid sites.

Total amount of acid sites: 0.99 mmol/g, as determined according to the NH₃-TPD method described in Reference Example 1.1.

Total amount of medium acid sites: 0.46 mmol/g, as determined according to the NH₃-TPD method described in Reference Example 1.1.

Total amount of strong acid sites: 0.02 mmol/g, as determined according to the NH₃-TPD method described in Reference Example 1.1.

### Reference Example 2.3

### Providing a Zeolitic Material having Framework Type BEA, a Molar Si:Al ratio of 12.5:1 and a Total Amount of Acid Sites of 1.019 mmol/g (Comparative)

The zeolitic material according to this Reference Example 2.3 is the zeolitic material CP814E as obtained from Zeolyst International. The zeolitic material CP814E has a molar Si:Al ratio of 12.5:1. Prior to use, this material was calcined in air at a temperature of 550° C. for 5 h, the heating rate to achieve this temperature was 2 K/min.

An NH₃-TPD analysis as disclosed in Reference Example 1.1 was performed. The NH₃-TPD plot is shown in FIG. 3: a weak peak was observed at the temperature of 590.2° C. which indicates that a small amount of strong acid sites is present in the zeolite. No peak of the medium acid sites was observed indicating the lack of medium acid sites in the zeolite. The peak observed at a temperature of 207.0° C. relates to weak acid sites. The integration of the curve between the temperatures of 100° C. and 600° C. gave the amount of the total acid sites.

Total amount of acid sites: 1.02 mmol/g, as determined according to the NH₃-TPD method described in Reference Example 1.1.

Total amount of medium acid sites: none detected according to the NH3-TPD method described in Reference Example 1.1.

Total amount of strong acid sites: 0.02 mmol/g, as determined according to the NH₃-TPD method described in Reference Example 1.1.

### Reference Example 2.4

### Preparation of a Zeolitic Material having Framework Type BEA, a Molar Si:Al ratio of 2.5:1 and a Total Amount of Acid Sites of 1.972 mmol/g (Comparative)

a) Preparation a an Al-Beta Zeolite

335.1 g of NaAlO₂ were dissolved in 7314 g of H₂O while stirring, followed by addition of 74.5 g of zeolite Beta seeds (commercially available from Zeolyst International, Valley Forge, Pa. 19482, USA, under the tradename CP814C, CAS Registry Number 1318-02-1). The mixture was placed in a 20 L autoclave and 7340 g sodium waterglass and 1436 g Ludox® AS40 were added. Crystallization of the obtained aluminosilicate gel took place at 120° C. for 117 h. After having let the reaction mixture cool to room temperature, the solid was separated by filtration, repeatedly washed with distilled water and then dried at 120° C. for 16 h. The resulting material had a water uptake of 12 weight-%.

b) Treatment with Ammonium Nitrate

Materials Used:

| | | |
|---|---|---|
| Distilled water | (2x) | 1,6110 g |
| ammonium nitrate (NH₄NO₃) 10% | (2x) | 179.0 g |
| Na-zeolite (powder) of a) | | 179.0 g |

The zeolite was prepared via ion exchange from the Na-form to the H-Form. 1,790 g of a 10% ammonium nitrate solution was prepared from distilled water and ammonium nitrate. While stirring, 179 g of Na-zeolite were added at a pH of 2.6 and heated to 80° C. for 2 h. After cooling, the suspension was filtered off and washed to neutral pH with 12 L of distilled water. The filter cake was stirred together with the 1,790 g of 10% ammonium nitrate solution for 2 h at 80° C. The suspension is filtered off and washed again to neutral pH with 12 L of distilled water. The entire ion exchange process was then repeated again. The zeolite was dried for 5 h at 120° C. and calcined at 500° C. for 5 h. 142 g of a calcinated beta-zeolite in its H-form were obtained. Elementary analysis: Al=7.1%; Si=38%; Na=0.07%.

An NH₃-TPD analysis as disclosed in Reference Example 1.1 was performed. The NH₃-TPD plot is reported in FIG. 4: a weak peak was observed at a temperature of 572.1° C. which indicates that a small amount of strong acid sites was present in the zeolite. No peak of the medium acid sites was observed indicating the lack of medium acid sites in the zeolite. The peak observed at a temperature of 229.9° C. relates to weak acid sites. The integration of the curve between the temperatures of 100° C. and 600° C. gave the amount of the total acid sites.

Total amount of acid sites: 1.97 mmol/g, as determined according to the NH₃-TPD method described in Reference Example 1.1.

Total amount of medium acid sites: none detected according to the NH₃-TPD method described in Reference Example 1.1.

Total amount of strong acid sites: 0.02 mmol/g, as determined according to the NH₃-TPD method described in Reference Example 1.1.

### Example 1

### General Procedure for the Preparation of a Lactide (Compound of Formula (II)

The preparation of the lactide was carried out in a series of individual experiments designated E1.1 to CE3 according to Table 1 below.

a) Continuous Mode

An aqueous solution of lactic acid (50 weight-%) was at least partially converted into the lactide of formula (II) according to the present invention in toluene. In first step a mixture was prepared by filling
50 g of toluene, and
2.5 g of the respective zeolitic material into a 100 mL three-necked round bottom flask operates as a continuous flow stirred-tank reactor (CSTR) with removal of the water at reflux temperature and with condenser and phase-separator for continuous toluene recirculation. In a second step the mixture inside the glass reactor was heated to reflux temperature while stirring. After the corresponding reaction temperature was reached, a specific constant flow of an aqueous solution of lactic acid (50-90 weight-%) was added. The flow and reaction temperature was maintained for 3 hours (see table 1, below) while continuing stirring the reaction mixture inside the heated glass reactor.

b) Batch Mode

An aqueous solution of lactic acid (50 weight%) was at least partially converted into the lactide of formula (II) according to the present invention) in toluene.

In first step a mixture was prepared by filling
10 g of toluene,
0.5 g of the respective zeolitic material, and
1.67 g of an aqueous solution of lactic acid (50 weight-%) into a 25 mL three-necked round bottom flask operates with removal of the water at reflux temperature and with condenser and phase-separator for continuous toluene recirculation.

In a second step the mixture inside the glass reactor was heated to reflux temperature while stirring. The reaction temperature was maintained for 3 hours (see Table 1, below) while continuing stirring the reaction mixture inside the heated glass reactor.

### Example 1.1

### Using the Zeolitic Material of Reference Example 2.1: Flow Rate of 46.39 microliter/min (E1.1)

The compound of formula (II) was prepared according to the general procedure disclosed above using the zeolitic material prepared in Reference Example 2.1 at a flow rate of 46.39 microliter/min.

### Example 1.2

### Using the Zeolitic Material According to Reference Example 2.1: Flow Rate of 23 microliter/min (E1.2)

The lactide was prepared according to the general procedure disclosed above using the zeolitic material prepared in Reference Example 2.1 at a flow rate of 23 microliter/min.

### Example 1.3

### Using the Zeolitic Material According to Reference Example 2.1: Batch Experiment (E1.3)

The lactide was prepared according to the general procedure disclosed above using the zeolitic material prepared in Reference Example 2.1. The experiment was carried out in batch mode.

### Example 1.4

### Using the Zeolitic Material According to Reference Example 2.2: Flow Rate of 46.39 microliter/min (E1.4)

The lactide was prepared according to the general procedure disclosed above using the zeolitic material prepared in Reference Example 2.2 at a flow rate of 46.39 microliter/min.

### Example 1.5

### Using the Zeolitic Material According to Reference Example 2.2: Batch Experiment (E1.5)

The lactide was prepared according to the general procedure disclosed above using the zeolitic material prepared in Reference Example 2.2. The experiment was carried out in batch mode.

### Comparative Example 1

### Preparation of Lactide Using the Zeolitic Material According to Reference Example 2.3: Flow Rate of 46.39 microliter/min (CE1)

The lactide was prepared according to the general procedure disclosed above in Example 1 using the zeolitic material prepared in Reference Example 2.3 at a flow rate of 46.39 microliter/min.

### Comparative Example 2

### Preparation of Lactide Using the Zeolitic Material According to Reference Example 2.4: Batch Experiment (CE2)

The lactide was prepared according to the general procedure disclosed above in Example 1 using the zeolitic material prepared in Reference Example 2.4. The experiment was carried out in batch mode. The results of the examples and the comparative examples are shown in Table 1 below.

TABLE 1
Results of the inventive examples E1.1 to E.1.5
and the comparative examples CE1 and CE2

| | operation mode | | | | | | |
|---|---|---|---|---|---|---|---|
| zeolitic | continous | continous | continous | continous | batch | batch | batch |
| material | E1.1 | E1.2 | E1.4 | CE1 | E1.3 | E1.5 | CE2 |
| Y₁/% ⁽¹⁾⁽⁷⁾ | 56.1 | 61.5 | 51.4 | 44.9 | n.d. ⁽⁸⁾ | n.d. ⁽⁸⁾ | n.d. ⁽⁸⁾ |
| comp. (II) | | | | | | | |
| Y₂/% ^{(1)(7/} | 51.0 | 55.5 | n.d. ⁽⁸⁾ | 36.2 | 83.3 | 75.1 | 17.5 |
| comp. (II) | | | | | | | |
| C₁/% ⁽¹⁾⁽⁷⁾ | 73.9 | 79.6 | 81.1 | 65.3 | n.d. ⁽⁸⁾ | n.d. ⁽⁸⁾ | n.d. ⁽⁸⁾ |
| comp. (I) | | | | | | | |
| C₂/% ⁽¹⁾⁽⁷⁾ | 81.5 | 78.0 | n.d. ⁽⁸⁾ | 62.8 | 98.8 | 96.5 | 43.5 |
| comp. (I) | | | | | | | |
| S₁/% ⁽¹⁾⁽⁷⁾ | 79.5 | 77.3 | 63.4 | 68.8 | n.d. ⁽⁸⁾ | n.d. ⁽⁸⁾ | n.d. ⁽⁸⁾ |
| comp. (II) | | | | | | | |
| S₂/% ⁽¹⁾⁽⁷⁾ | 62.6 | 71.2 | n.d. ⁽⁸⁾ | 57.6 | 84.3 | 77.8 | 31.0 |
| comp. (II) | | | | | | | |

⁽¹⁾ Y₁: yield of the compound (II) at a contacting time in (ii) of 2 h
⁽²⁾ Y₂: yield of the compound (II) at a contacting time in (ii) of 3 h
⁽³⁾ C₁: conversion of the compound (I) at a contacting time in (ii) of 2 h
⁽⁴⁾ C₂: conversion of the compound (I) at a contacting time in (ii) of 3 h
⁽⁵⁾ S₁: selectivity of the compound (II) at a contacting time in (ii) of 2 h
⁽⁶⁾ S₂: selectivity of the (II) at a contacting time in (ii) of 3 h
⁽⁷⁾ Yield Y/% is defined as [mmol of compound (II) obtained from (ii)/mmol of compound (I) subjected to (i)]*100
Conversion C/% is defined as [(mmol of compound (I) obtained from (ii)/mmol of compound (I) subject to (i)]*100
Selectivity S/% is defined as (Y/C)*100
⁽⁸⁾ not determined

As can be taken from the results according to Table 1 above, the inventive use of the zeolitic materials of examples E1.1 to E1.5 results in a higher yield compared to use of the zeolitic material of comparative examples CE1 and CE2. Further, the inventive use of the zeolitic materials of examples E1.1 to E1.5 leads to a higher conversion compared to the use of the zeolitic materials of comparative example CE1 to CE2. Therefore, it is shown that—although in a particular case of the zeolitic material of E.1.4, a slightly lower selectivity is observed compared to the zeolitic material of CE1, all zeolitic materials of examples E1.1 to E1.5 are more active than the zeolitic material of comparative examples CE1 and CE2 and, thus, lead to a higher productivity since more product per time is produced.

### SHORT DESCRIPTION OF THE FIGURES

FIG. 1: shows the NH₃-TPD plot of the zeolite material of Reference Example 2.1 as measured according to Reference Example 1.1: no peak is observed at a temperature above 500° C. indicating that no strong acid site is present in the zeolite. A peak is observed at 328.9° C. indicating the presence of medium acid sites in the zeolite. The peak observed at a temperature of 177.8° C. is relative to the weak acid sites. The integration of the curve between the temperatures of 100° C. and 550° C. gives the amount of the total acid sites.

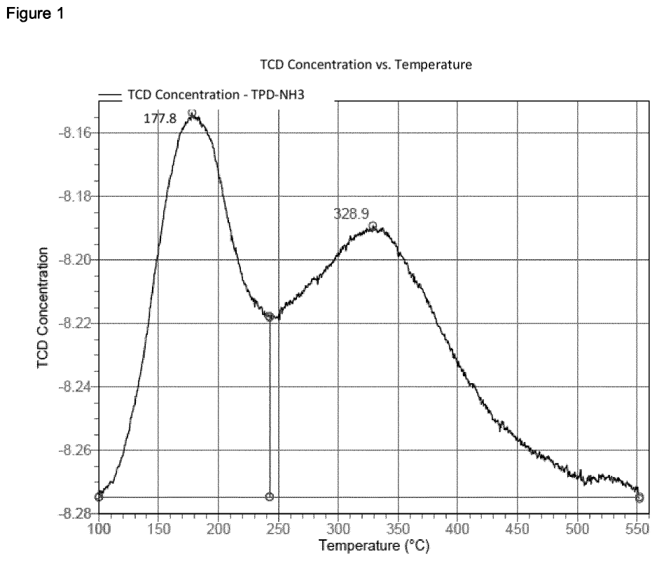

FIG. 2: shows the NH₃-TPD plot of the zeolite material of Reference Example 2.2 as measured according to Reference Example 1.1: a weak peak is observed at a temperature of 594.3° C. indicating that a small amount of strong acid sites is present in the zeolite. A peak is observed at 374.1° C. indicating the presence of medium acid sites in the zeolite. The peak observed at a temperature of 223.8° C. is relative to the weak acid sites. The integration of the curve between the temperatures of 100° C. and 600° C. gives the amount of the total acid sites.

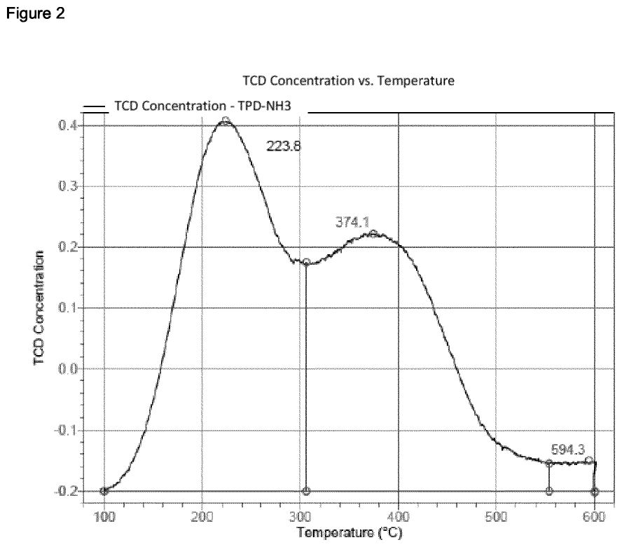

FIG. 3: shows the NH₃-TPD plot of the zeolite material of Reference Example 2.3 as measured according to Reference Example 1.1: a weak peak is observed at the temperature of 590.2° C. indicating that a small amount of strong acid sites is present in the zeolite. No peak of the medium acid sites is observed indicating the lack of medium acid sites in the zeolite. The peak observed at a temperature of 207.0° C. is relative to the weak acid sites. The integration of the curve between the temperatures of 100° C. and 600° C. gives the amount of the total acid sites.

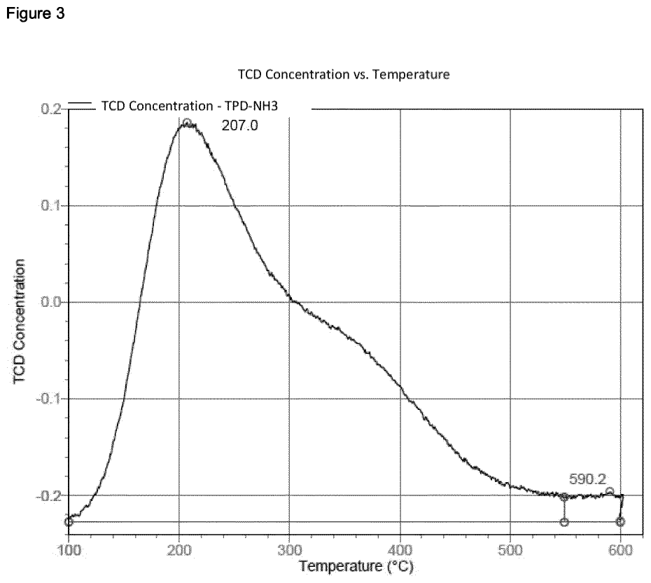

FIG. 4: shows the NH₃-TPD plot of the zeolite material of Reference Example 2.4 1as measured according to Reference Example 1.1: a weak peak is observed at a temperature of 572.1° C. indicating that a small amount of strong acid sites are present in the zeolite. No peak of the medium acid sites is observed indicating the lack of medium acid sites in the zeolite. The peak observed at a temperature of 229.9° C. is relative to the weak acid sites. The integration of the curve between the temperatures of 100° C. and 600° C. gives the amount of the total acid sites.

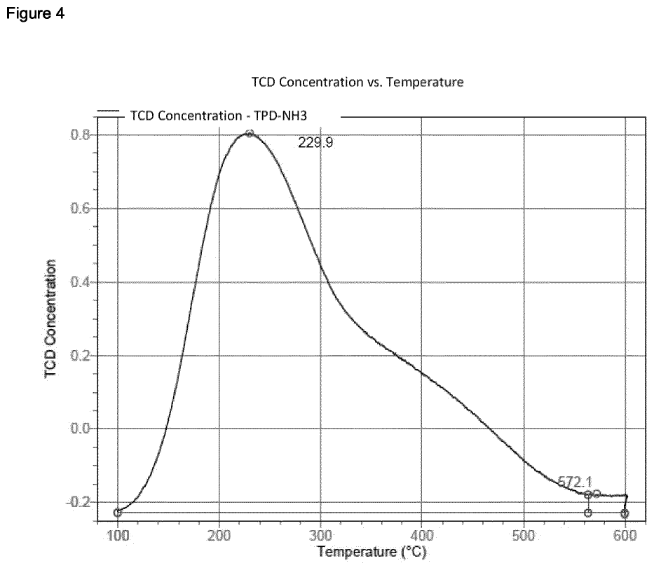

### CITED PRIOR ART

WO 2014/122294 A
WO 2010146156 A
WO 2014/060260 A
Dusselier et al., Supplementary Materials for “Shape-selective zeolite catalysis for bioplastics production”, www.sciencemag.org/content/349/6243/78/suppl/DC1 Published 3 Jul. 2015, Science, 349, 78 (2015) DOI: 10.1126/science.aaa7169

1. A process for preparing a compound of formula (II) 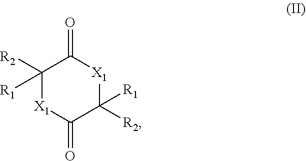
comprising
(i) providing a mixture comprising a compound of formula (I) 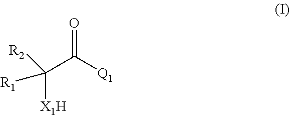
or a salt thereof;
(ii) contacting the mixture provided in (i) with a catalyst comprising a zeolitic material, obtaining a mixture (ii) comprising the compound of formula (II);
wherein
X₁ is O or NH,
R₁ and R₂ are, independently of each other, H, C₁-C₁₀ alkyl, C₂-C₁₀ alkenyl, C₂-C₁₀ alkynyl, or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;
Q₁ is OH, OR₃, NH₂, Cl, Br, or I;
R₃ is C₁-C₁₀ alkyl or C₆-C₁₂ aryl, each being optionally substituted by one or more of C₁-C₆ alkyl, C₁-C₆ alkyloxy, C₂-C₆ alkenyl, C₂-C₆ alkynyl, and C₆-C₁₂ aryl;
wherein the zeolitic material in (ii) has framework type BEA and wherein the framework structure of the zeolitic material comprises Si, Al, O, and H;
wherein the zeolitic material has a total amount of acid sites in the range of from 0.25 to 1.0 mmol/g, wherein the total amount of acid sites is defined as the total molar amount of desorbed ammonia per mass of the zeolitic material determined according to the temperature programmed desorption of ammonia;
wherein the zeolitic material has an amount of medium acid sites wherein the amount of medium acid sites is defined as the amount of desorbed ammonia per mass of the zeolitic material determined according to the temperature programmed desorption of ammonia;
wherein the amount of medium acid sites is at least 40% of the total amount of acid sites.

2. The process of claim 1, wherein the amount of medium acid sites is in the range of from 40 to 70%.

3. The process of claim 1, wherein the amount of strong acid sites of the zeolitic material defined as the amount of desorbed ammonia per mass of the zeolitic material determined according to the temperature programmed desorption of ammonia in the temperature range above 500° C. is in the range of from 0 to 0.10 mmol/g.

4. The process of claim 1, wherein the framework structure of the zeolitic material has a molar ratio Si:Al in the range of from 15:1 to 30:1.

5. The process of claim 1, wherein the zeolitic material comprised in the catalyst according to (ii) is obtained by an organotemplate-free synthesis method, said organotemplate-free synthesis method comprising:
(1) providing a mixture comprising one or more sources for SiO₂, one or more sources for Al₂O₃, and seed crystals, wherein the seed crystals comprise a zeolitic material having framework type BEA;
(2) crystallizing the mixture obtained in step (1), obtaining a mixture comprising the zeolitic material having a framework type BEA;
(3) isolating the zeolitic material having framework type BEA from the mixture obtained from (2);
(4) optionally drying and calcining the zeolitic material having framework type BEA; and
(5) optionally subjecting the zeolitic material obtained from (3) or (4), by a method comprising
(5.1) treating the zeolitic material with an aqueous solution having a pH of at most 5;
(5.2) treating the zeolitic material obtained from (5.1) with a liquid aqueous system having a pH in the range of 5.5 to 8 and a temperature of at least 75° C.;
wherein after (5.2), the zeolitic material is optionally subjected to at least one further treatment according to (5.1) and/or at least one further treatment according to (5.2).

6. The process of claim 1, wherein the mixture provided in (i) further comprises water, wherein in the mixture provided in (i), the weight ratio of the compound of formula (I) relative to the water is in the range of from 95:5 to 45:55.

7. The process of claim 1, wherein the mixture provided in (i) further comprises an organic solvent, wherein in the mixture provided in (i), the molar ratio of the compound of formula (I) relative to the organic solvent is in the range of from 0.01:1 to 3:1.

8. The process of claim 1, wherein the mixture provided in (i) is provided in liquid phase and wherein according to (ii), the mixture provided in (i) is contacted with the catalyst in liquid phase or in gas phase.

9. The process of claim 1, wherein the contacting of (ii) is carried out under water removal conditions, wherein the contacting in (ii) is carried out at a temperature of the mixture brought in contact with the catalyst of at least 100° C., and wherein the contacting in (ii) is carried out at a pressure of the mixture brought in contact with the catalyst in the range of from 2 to 10 bar_{(abs)}.

10. The process of claim 1, wherein the contacting in (ii) is carried out in continuous mode, at a weight hourly space velocity in the range of from 0.5 to 10 h⁻¹, wherein the weight hourly space velocity is defined as the mass flow rate of the compound of formula (I) comprised in the mixture provided in (i) and subjected to (ii) in kg/h divided by the mass of the zeolitic material comprised in the catalyst in kg with which the mixture provided in (i) is contacted in (ii).

11. The process of claim 1, further comprising separating the compound of formula (II) from the mixture obtained from (ii).

12. The process of claim 1, wherein the mixture obtained in (ii) further comprises the compound of formula (I), the process further comprising:
recycling the compound of formula (I), and
recycling the organic solvent.

13. The process of claim 1, wherein C₁-C₁₀-alkyl is methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methyl-propyl, n-heptyl, or n-octyl.

14. The process of claim 1, wherein the compound of formula (I) is one or more of 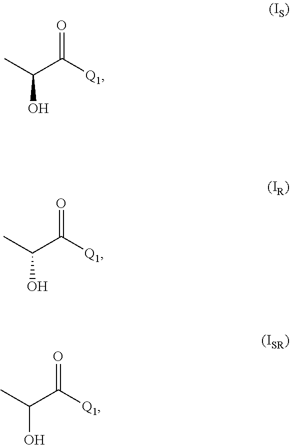
and wherein the compound of formula (II) is one or more of 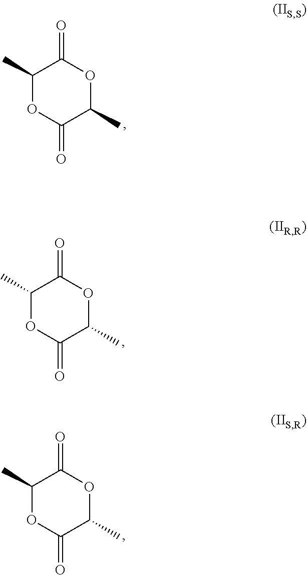
and the racemate of (II_{S,S}) and (II_{R,R}).

15. A mixture, obtained by a process according to claim 1, comprising a compound of formula (II). [chemical structure]
